(19) United States
(12) Patent Application Publication
(10) Pub. No.: US 2007/0134230 A1
(43) Pub. Date: Jun. 14, 2007

(54) METHOD FOR PROLONGING ACTIVITY OF AUTODEGRADABLE ENZYMES

(76) Inventors: Dharmendra M. Jani, Fairport, NY (US); Afshin Shafiee, Rochester, NY (US); Bruce A. Pfeffer, Fairport, NY (US); Michael K. Hartzer, Arlington, TX (US)

Correspondence Address: Toan P. Vo;Bausch & Lomb Incorporated, One Bausch & Lomb Place, Rochester, NY, 14604, US

(21) Appl. No.: 11/567,338

(22) Filed: Dec. 6, 2006

Related U.S. Application Data
(60) Provisional application No. 60/750,031, filed on Dec. 13, 2005.

(51) Int. Cl. A61K 38/48; C12N 9/64

(52) U.S. Cl. 424/94.63; 435/226

(57) ABSTRACT

A method for prolonging the activity of an autodegradable enzyme comprises storing the enzyme after manufacture at a pH less than about 5, and reconstituting the acidified enzyme substantially immediately before use with a buffer having a pH in the range from about 6.5 to about 11, wherein the pH remains within 1 pH unit upon adding said the enzyme into the buffer. The method is useful to provide enzyme for wide use, which otherwise would lose activity upon long storage. In one embodiment the method is applicable to provide enzyme for inducing controlled posterior vitreous detachment.

### CROSS-REFERENCE OF RELATED APPLICATION

This application claims the benefit of U.S. Provisional Patent Application No. 60/750,031 filed Dec. 16, 2006, and is incorporated herein by reference.

### BACKGROUND OF THE INVENTION

The present invention relates to a method for prolonging the activity of autodegradable enzymes. In particular, the present invention relates to a method for prolonging the enzymatic activity of plasmin or derivatives thereof. More particularly, the present invention relates to a method for obtaining extended in-vivo enzymatic activity of plasmin or derivatives thereof after storage and to a method for effecting posterior vitreous detachment using such plasmin or derivatives thereof.

Proteases (or proteolytic enzymes or peptidases) are enzymes that catalyze the degradation or breakdown of proteins and, thus, participate in many important physiologic processes. A protease or peptidase can be further classified as an endopeptidase (which cleave peptide bonds within a protein) or exopeptidase (which removes amino acid sequentially from either the N- or the C-terminus of a protein). An endopeptidase is also termed a “proteinase.” Plasmin, a serine proteinase, is the principal fribrinolytic enzyme in mammals, and has the important function of breaking down in-vivo blood clots. It derives from the inactive precursor plasminogen, which circulates in plasma at a concentration of about 1.5 μM. Circulating plasminogen is activated, for example in vivo, by plasminogen activators, such as tissue plasminogen activator (“tPA”) or urokinase, which cleave a single-chain plasminogen molecule at the Arg⁵⁶⁰-Val⁵⁶¹ peptide bond, producing active plasmin. Plasminogen is also activatable by the bacteria-derived enzyme streptokinase. Thus, thrombolytic drugs, such as those based on tPA, streptokinase, and urokinase, have been developed for administering into patients suffered from various thrombotic disorders, including myocardial infarction, occlusive stroke, deep venous thrombosis, and peripheral arterial disease, to promote the in-vivo production of plasmin in order rapidly to enhance the degradation of blood clots. However, the administered tPA, streptokinase, or urokinase still must encounter the circulating plasminogen in order to generate active plasmin, and the magnitude of their effectiveness still depends on the inherent in-vivo level of plasminogen. Therefore, it has been thought that a higher benefit should be obtained if active plasmin is administered instead into these patients.

Plasmin also has been proposed for inducing controlled posterior vitreous detachment (“PVD”) to prevent, stop, or reduce the progression of retinal detachment.

The vitreous is a clear, proteinaceous mass which fills the posterior cavity of the eye between the lens and the retina. The vitreous is attached at its posterior face to the retina along the structure known as the internal limiting membrane. This site of attachment of the vitreous and the retina is termed the vitreoretinal junction and consists of a layer of basement membrane proximal to the retina and a layer of collagen fibrils proximal to the vitreous.

Degenerative changes in the vitreous are a precursor to posterior vitreous detachment (“PVD”). Degeneration of the vitreous is part of the normal aging process, but also may be induced by pathological conditions such as diabetes, Eales' disease and uveitis (Gloor, B. P., “The Vitreous”, in Adler's Physiology of the Eye, C. V. Mosby, St. Louis, Mo., 1987). Because the vitreous is attached to the retina, the receding vitreous can cause a retinal tear, with subsequent detachment of the retina.

Certain pathological conditions of the eye are accompanied by the formation of new (abnormal) vessels on the surface of the retina—namely proliferative diseases. With a PVD, traction is placed on new vessels causing rupture and bleeding. Proliferative retinal diseases thus are accompanied by both a high probability of retinal detachment as well as complications from bleeding resulting from the rupture of the newly formed blood vessels. Thus, it is beneficial to induce a controlled PVD before damage to the retina occurs because of uncontrolled detachment.

Verstraeten et al. (Arch. Ophthalmol., Vol. 11, 849-854 (1993)) proposed the use of plasmin to produce a cleavage at the vitreoretinal interface. Plasmin hydrolyzes glycoproteins, including laminin and fibronectin, which are found at the vitreoretinal junction. Plasmin treatment was performed with or without subsequent vitrectomy on rabbit eyes. The authors noted that eyes treated with plasmin showed some areas of PVD, but only after vitrectomy was the vitreous substantially detached. The authors concluded that plasmin treatment may be useful as a biochemical adjunct to mechanical vitrectomy. However, plasmin rapidly autodegrades at or near physiological pH, at which it has the highest activity, and has not been available for therapeutic administration, as it cannot be stored at this pH. Therefore, U.S. Pat. No. 6,355,243, for example, teaches that isolated plasmin is stored at pH less than about 4 to avoid its autodegradation. However, when plasmin at such a low pH is administered into a patient whose physiological pH is about 7.4, undesirable effects may occur.

Therefore, there is a need to provide a method for obtaining enzymes having activity at or near that at the time of its manufacture, after a prolonged storage. In addition, it is also desirable to provide a method for prolonging the activity of an enzyme after it has been administered into a patient. Moreover, it is also desirable to provide a method for stabilizing plasmin and derivatives thereof during storage, regaining their activity when they are ready for use, and prolonging their activity in vivo after administration into a patient.

### SUMMARY OF THE INVENTION

In general, the present invention provides a method for producing an active enzyme after prolonged storage, the method comprising: (a) storing said enzyme at a pH less than about 5; and (b) adding said enzyme to a buffer having pH corresponding approximately to a pH at which said enzyme has the highest activity in a preselected reaction or use, to produce a buffered enzyme substantially immediately before using the enzyme or carrying out the reaction, wherein said enzyme is autodegradable at pH greater than about 5; and said buffer has a capacity such that the pH of the buffer changes by less than about 1 pH unit upon adding said enzyme.

In one aspect, the enzyme is a proteolytic enzyme (or alternatively termed “protease,” or “peptidase”).

In another aspect, the enzyme is selected from the group consisting of serine proteinases, cysteine proteinases, aspartyl proteinases, metalloproteinases (or alternatively termed “matrix metalloproteinases”), combinations thereof, and mixtures thereof.

In still another aspect, the present invention provides a method for prolonging an activity of an enzyme at physiological pH, which enzyme is autodegradable at a physiological pH, the method comprising: (a) providing said enzyme that have been preserved at a pH less than about 5; and (b) adding said enzyme to a buffer having approximately physiological pH to produce a buffered enzyme before administering said buffered enzyme into a patient, thereby prolonging the activity of said enzyme in said patient, wherein the post-administering enzyme activity is higher than the activity of unbuffered enzyme in said patient.

In yet another aspect, the present invention provides a method for preventing or reducing a precipitation of an enzyme administered into a vitreous of an eye, the method comprising: (a) providing the enzyme at a pH of less than about 5; (b) adding said enzyme to a buffer having a pH in a range from about 6.5 to about 8 to produce a buffered enzyme before administering said buffered enzyme into the vitreous of the eye, wherein upon adding the enzyme to the buffer, the pH of the buffer remains within about 1 pH unit of the pH of the buffer.

In a further aspect, the present invention provides a method for inducing PVD in an eye, the method comprising: (a) providing plasmin or derivatives thereof that have been preserved at a pH less than about 5; and (b) adding said plasmin or derivatives thereof to a buffer having a pH in a range from about 6.5 to about 11 to produce a buffered plasmin or derivatives thereof before administering said buffered plasmin or derivatives thereof into a posterior chamber of the eye, thereby inducing PVD in said eye.

Other features and advantages of the present invention will become apparent from the following detailed description and claims and the appended drawings.

### BRIEF DESCRIPTION OF THE DRAWINGS

FIG. 1 shows the pH dependence of the enzymatic activity of plasmin, as measured by the hydrolysis of peptidase substrate S-2251.

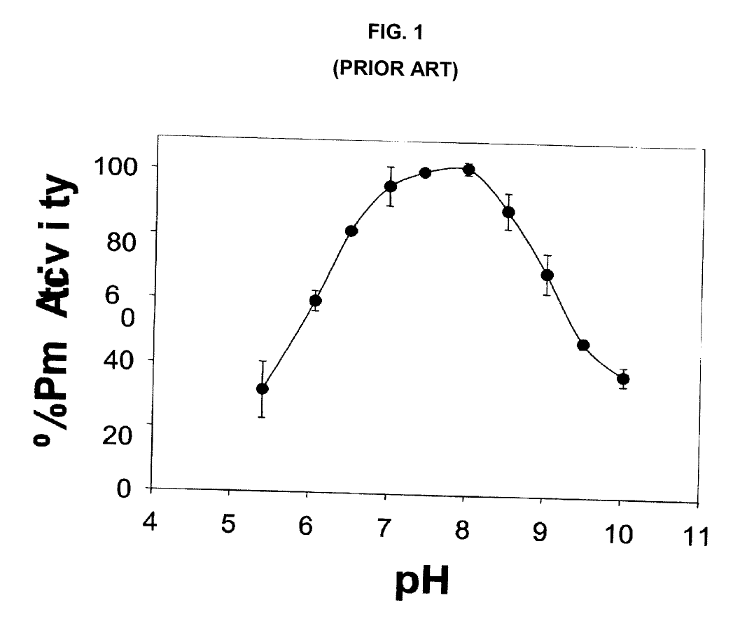

FIG. 2 shows the relative activity of reconstituted plasmin in buffered and saline solutions.

### DETAILED DESCRIPTION OF THE INVENTION

As used herein, the terms “autodegradable enzyme” and “autolyzable enzyme” are used interchangeably and means an enzyme that is capable of breaking down, digesting, degrading, or hydrolyzing its own molecules due to its enzymatic or catalytic activity. The term “physiological pH” means pH of about 7.4.

In general, the present invention provides a method for producing an active enzyme after prolonged storage, the method comprising: (a) storing said enzyme at a pH less than about 5; and (b) adding said enzyme to a buffer having pH corresponding approximately to a pH at which said enzyme has the highest activity in a preselected reaction or use, to produce a buffered enzyme substantially immediately before using the enzyme or carrying out the reaction, wherein said enzyme is autodegradable at pH greater than about 5; and said buffer has a capacity such that the pH of the buffer changes by less than about 1 pH unit upon adding said enzyme.

In one aspect, said buffer pH changes by less than about 0.5 (or, alternatively, about 0.2, or about 0.1) pH unit upon adding said enzyme. The relative amounts of the enzyme and the buffer are thus chosen based on the desired maximum change in the pH of the solution, the enzyme, and the type of buffer.

In another aspect, the step of storing of said enzyme is effected at a pH less than about 4. Alternatively, said pH is less than 3.5 or in the range from about 2.5 to about 4, or from about 2.5 to about 3.5, or from about 3 to about 3.5.

The present invention is useful in producing an active enzyme after prolonged storage after its manufacture. Such an enzyme is reconstituted in a composition and is available for use, such as a therapeutic or diagnostic use, after a prolonged storage. In particular, the present invention provides a solution to the problem of decay of activity of autodegradable enzymes upon storage, which have not been adopted for wide use because of such autodegradation or autolysis.

In one aspect, the enzyme is a proteolytic enzyme. In another aspect, the enzyme is selected from the group consisting of serine proteinases, cysteine proteinases, aspartyl proteinases, metalloproteinases, combinations thereof, and mixtures thereof. In still another aspect, the enzyme is selected from the group consisting of serine proteinases. Non-limiting examples of such serine proteinases include plasmin, trypsin, chymotrypsin, elastase, carboxypeptidase, and combinations thereof.

As used herein, “derivatives” of an enzyme encompass variants of the enzyme that still substantially retain the basic enzymatic function of the enzyme. Such variants can be modified forms of the enzyme, such as for example a truncated form wherein one or more amino acid residues or segments of the enzyme molecule are deleted. Such variants also can be a form of the enzyme wherein one or more amino acid residues are substituted, such as by conservative substitutions, or wherein one or more amino acid residues are added to the polypeptide. In one aspect, the enzyme is plasmin or a derivative thereof. As used herein, a derivative of plasmin encompasses a polypeptide that is a fragment or portion thereof that can comprise the enzymatic or catalytic domain or region of plasmin. A derivative of plasmin can further comprise a kringle domain or region of the plasmin molecule. A kringle domain of plasmin is characterized by a triple-loop conformation and comprises about 75-85 amino acid residues with three disulfide bridges. Within the scope of derivatives of plasmin is microplasmin, which comprises the serine proteinase enzymatic domain of plasmin and a short polypeptide sequence (e.g., comprising about 25-40 amino acid residues) between the enzymatic domain and the kringle-5 domain of plasmin.

In another aspect, a derivative of plasmin can be a miniplasmin, which comprises the kringle-5 domain and the enzymatic domain of plasmin. Enzymatically active microplasmin and miniplasmin are obtained from microplasminogen and miniplasminogen precursors by cleavage of the peptide bond at Arg⁵⁶¹-Val⁵⁶², wherein the amino acid residue numbers correspond to those of human Glu-plasminogen, which has 791 amino acid residues. Microplasminogen and miniplasminogen are disclosed in U.S. Patent Application Publications 2004/0071676 A1 and 2005/0124036 A1, which are incorporated herein by reference in their entirety.

Plasmin can be produced by activation of plasminogen precursor, which may be obtained from plasma. For example, a method of producing high-purity plasmin is disclosed in U.S. Patent Application Publication 2004/0171103 A1, which is incorporated herein by reference in its entirety. The starting material, plasminogen, can be extracted from Cohn Fraction II+III paste by affinity chromatography on Lys-SEPHAROSE™ as described by D. G. Deutsch and E. T. Mertz, “Plasminogen: purification from human plasma by affinity chromatography,” Science 170(962):1095-6 (1970). (SEPHAROSE™ is a trade name of Pharmacia, Inc., New Jersey.)

Following the extraction of plasminogen from the Cohn Fraction II+III paste, lipid and protein impurities and Transmissible Spongiform Encephalopathies (“TSE”) contaminants are reduced by precipitation with the addition polyethylene glycol (“PEG”), in a range of about 1 to about 10 percent weight/volume or the addition of about 80 to about 120 g/l ammonium sulfate. The PEG or ammonium sulfate precipitate is removed by depth filtration and the resulting solution placed on a lysine affinity resin column. The phrase “lysine affinity resin” is used generally for affinity resins containing lysine or its derivatives or ε-aminocaproic acids as the ligand. The column can be eluted with a low pH solution of approximately 1 to 4.

The protein obtained after elution from the affinity column is generally at least 80 percent plasminogen. The purified plasminogen is then stored at low pH in the presence of simple buffers such as glycine and lysine or ω-amino acids.

Plasminogen in solution is then activated to plasmin by the addition of a plasminogen activator, which may be accomplished in a number of ways including but not limited to streptokinase, urokinase, tissue plasminogen activator (“tPA”), or the use of urokinase immobilized on resin and use of streptokinase immobilized on resin. In one embodiment, the plasminogen activator is soluble streptokinase. The addition of stabilizers or excipients such as glycerol, ω-amino acids such as lysine, polylysine, arginine, ε-aminocaproic acid and tranexamic acid (trans-4-(aminomethyl)cyclohexanecarboxylic acid), and salt enhances the yield of plasmin.

Plasmin can be purified from unactivated plasminogen by affinity chromatography on resin with benzamidine as the ligand and eluted preferably with a low pH solution (e.g., pH<4, or alternatively pH between about 2.5 and about 4). This step can remove essentially all degraded plasmin as well as the majority of the streptokinase.

As a polishing step for the removal of remaining streptokinase, hydrophobic interaction chromatography (“HIC”) at low pH is performed (e.g., pH<4). Following the HIC step, plasmin is formulated as a sterile protein solution by ultrafiltration and diafiltration and 0.22-μm filtration.

The eluted plasmin from such polishing step can be buffered with a low pH (e.g., pH<4), low buffering capacity agent. The low pH, low buffering capacity agent typically comprises a buffer of either an amino acid, a derivative of at least one amino acid, an oligopeptide which includes at least one amino acid, or a combination thereof. In addition, the low pH, low buffering capacity agent can comprise a buffer selected from acetic acid, citric acid, hydrochloric acid, carboxylic acid, lactic acid, malic acid, tartaric acid, benzoic acid, serine, threonine, methionine, glutamine, alanine, glycine, isoleucine, valine, alanine, aspartic acid, derivatives, and combinations thereof. The concentration of plasmin in the buffered solution can range from about 0.01 mg/ml to about 50 mg/ml of the total solution. The concentration of the buffer can range from about 1 nM to about 50 mM. Of course, these ranges may be broadened or narrowed depending upon the buffer chosen, or upon the addition of other ingredients such as additives or stabilizing agents. The amount of buffer added is typically that which will give the reversibly inactive acidified plasmin solution a pH between about 2.5 to about 4, or between about 3 and about 3.5.

It may be advantageous to add a stabilizing or bulking agent to the reversibly inactive acidified plasmin solution obtained as disclosed above. Non-limiting examples of such stabilizing or bulking agents are a polyhydric alcohols, pharmaceutically acceptable carbohydrates, salts, glucosamine, thiamine, niacinamide, and combinations thereof. The stabilizing salts can be selected from the group consisting of sodium chloride, potassium chloride, magnesium chloride, calcium chloride, and combinations thereof. Sugars or sugar alcohols may also be added, such as glucose, maltose, mannitol, sorbitol, sucrose, lactose, trehalose, and combinations thereof. Other carbohydrates that may be used are polysaccharides, such as dextrin, dextran, glycogen, starches, carboxymethylcellulose, derivatives thereof, and combinations thereof. Concentrations of a carbohydrate added to add bulk to the reversibly inactive acidified plasmin solution can be in a range from about 0.2 percent weight/volume (“% w/v”) to about 20% w/v. Concentrations for a salt, glucosamine, thiamine, niacinamide, and their combinations can range from about 0.01 M to about 1 M.

Plasmin formulated according to the method disclosed above in buffered acidified water has been found to be very stable. It can be kept in this form for months without substantial loss of activity or the appearance of degradation products of a proteolytic or acidic nature. At 4° C., such plasmin is stable for at least nine months. Even at room temperature, such plasmin is stable for at least two months.

Inactive acidified plasmin compositions including a bulking agent, such as a carbohydrate, can be optionally lyophilized at a temperature in a range, for example, from about 0° C. to about −50° C., or preferably from about 0° C. to about −20° C., to produce a powder for long-term storage.

In another aspect, plasmin or variants thereof can be produced by recombinant technology, and a method of the present invention is applied to such plasmin and variants thereof. For example, the production of recombinant microplasminogen (which can be activated to microplasmin by cleavage of the peptide bond at Arg⁵⁶¹-Val⁵⁶² using one of the plasminogen activators disclosed above) in the Pichia pastoris yeast system is disclosed in U.S. Patent Application Publication 2004/0071676 A1, which is incorporated herein by reference. Plasminogen and miniplasminogen (which also can be activated to miniplasmin by cleavage of the peptide bond at Arg⁵⁶¹-Val⁵⁶² using one of the plasminogen activators disclosed above) in the Pichia pastoris yeast system is disclosed in U.S. Patent Application Publication 2005/0124036 A1, which is incorporated herein by reference.

Recombinant plasmin or variants thereof are acidified and stored at pH less than about 5 (or alternatively less than 4, or between about 2.5 and about 3.5). The acidified enzyme is reconstituted by adding said enzyme to a buffer having pH corresponding approximately to a pH at which said enzyme has the highest activity in a preselected reaction or use, to produce a buffered enzyme substantially immediately before using the enzyme or carrying out the reaction; wherein said buffer has a capacity such that the pH of the buffer changes by less than about 1 pH unit upon adding said enzyme. In one embodiment, said buffer has a capacity such that the pH of the buffer changes by less than about 0.5 (or, alternatively, about 0.2, or about 0.1) pH unit upon adding said enzyme. In one aspect, the pH of the final buffered enzyme is not within about 0.2 pH unit of the isoelectric point of the enzyme.

In one aspect, the buffer has a pH of about 7. Alternatively, the buffer has a pH in a range from about 7 to about 7.5.

In another aspect, the buffer has a pH of about 7.4.

In still another aspect, the buffer is a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10×phosphate buffer saline (“PBS”) or 5×PBS solution.

In a further aspect, the present invention provides a method for prolonging an activity of plasmin or derivatives thereof in a posterior chamber of an eye, the method comprising: (a) providing said plasmin or derivatives thereof that have been preserved at a pH less than about 5; and (b) adding said plasmin or derivatives thereof to a buffer having a pH in a range from about 6.5 to about 11 to produce a buffered plasmin or derivatives thereof before administering said buffered plasmin or derivatives thereof into the posterior chamber of the eye, thereby prolonging the activity of plasmin or derivatives thereof in said posterior chamber of the eye; wherein the post-administering activity is higher than the activity of unbuffered plasmin or derivatives thereof in said posterior chamber of the eye, and said buffer has a capacity such that a pH of buffered solution of said plasmin or derivatives thereof remains within about 1 (or, alternatively, about 0.5, or about 0.2, or about 0.1) pH unit upon adding said plasmin or derivatives thereof. The activity of plasmin or derivatives thereof reconstituted in such a buffer when administered into the posterior chamber will decay more slowly than that of plasmin or derivatives thereof reconstituted in a non-buffer, such as saline solution.

In another aspect, the buffer has a pH in a range from about 6.5 to about 9, or alternatively, from about 6.5 to about 8.

In another aspect, the method of the present invention has an advantage of substantially preventing a precipitation of plasmin or derivatives thereof in the posterior chamber of the eye upon administering said plasmin or derivatives thereof.

In still another aspect, the present invention provides a kit for making an active enzyme or derivatives thereof. The kit comprises: (a) the enzyme or derivatives thereof that have been preserved at a pH less than about 5; and (b) a buffer having a pH in a range from about 6.5 to about 11, provided in a separate container or package. In one embodiment, the preserved enzyme or derivatives thereof are added to the buffer to produce the active enzyme or derivatives thereof substantially at the time of use. In another embodiment, said buffer has a capacity such that a pH of a buffered solution of said plasmin or derivatives thereof remains within about 1 (or, alternatively, about 0.5, or about 0.2, or about 0.1) pH unit upon adding said plasmin or derivatives thereof. In still another embodiment, said buffer has a pH in a range from about 6.5 to about 8.

In still another aspect, the present invention provides a method for inducing PVD in an eye, the method comprising: (a) providing plasmin or derivatives thereof that have been preserved at a pH less than about 5; and (b) adding said plasmin or derivatives thereof to a buffer having a pH in a range from about 6.5 to about 11 (or alternatively, from about 6.5 to about 8) to produce a buffered plasmin or derivatives thereof before administering said buffered plasmin or derivatives thereof into a posterior chamber of the eye, thereby inducing PVD in said eye, wherein said buffer has a capacity such that a pH of a buffered solution of said plasmin or derivatives thereof remains within about 1 (or, alternatively, about 0.5, or about 0.2, or about 0.1) pH unit upon adding said plasmin or derivatives thereof.

In still another aspect, the method of the present invention has an advantage of substantially preventing precipitation of said plasmin or derivatives thereof in said posterior chamber of the eye upon administering said plasmin or derivatives thereof.

In a further aspect, the present invention provides a method of for inducing PVD in an eye, the method comprising administering a formulation of plasmin or derivatives thereof into a posterior chamber of an eye of a patient in need of having PVD; wherein said plasmin or derivatives thereof have been preserved at a pH less than about 5; and the preserved plasmin or derivatives thereof have been added to a buffer having a pH in a range from about 6.5 to about 11 to produce said formulation of plasmin or derivatives thereof before administering said formulation into a posterior chamber of the eye, thereby inducing PVD in said eye, wherein said buffer has a capacity such that a pH of a buffered solution of said plasmin or derivatives thereof remains within about 1 (or, alternatively, about 0.5, or about 0.2, or about 0.1) pH unit upon adding said plasmin or derivatives thereof. In one embodiment, said plasmin or derivatives thereof are added to said buffer substantially immediately before said administering. In another embodiment, said formulation is administered in an amount containing a therapeutically effective amount of plasmin or derivatives thereof to induce said PVD.

Method of injecting plasmin or derivatives thereof into eye for PVD is now described.

Plasmin or derivatives thereof, which is reconstituted substantially immediately before administering into a patient with a buffer as disclosed above, can be injected intravitreally, for example through the pars plana of the ciliary body, to induce controlled PVD using a fine-gauge needle, such as 25-30 gauge. Administration of plasmin or derivatives thereof can be used to prevent, treat, or ameliorate the blinding complications of an ocular condition, such as diabetic retinopathy, central vein occlusion, proliferative vitreoretinopathy, or proliferative vascular retinopathy. Typically, an amount from about 25 μl to about 100 μl of a formulation comprising about 4-5 IU of plasmin or derivatives thereof per 50 μl of formulation is administered into the vitreous. Such administration of plasmin or derivatives thereof may be periodically repeated upon assessment of the treatment results and recommendation by a skilled medical practitioner.

In yet another aspect, the present invention provides a method for preventing or reducing a precipitation of an enzyme administered into a region of a patient, the method comprising: (a) providing the enzyme at a pH of less than about 5; (b) adding said enzyme to a buffer having a pH in a range from about 6.5 to about 11 to produce a buffered enzyme before administering said buffered enzyme into said region of the patient; wherein upon adding the enzyme to the buffer, the pH of the buffer remains within about 1 (or, alternatively, about 0.5, or about 0.2, or about 0.1) pH unit of the original buffer pH. In one embodiment of the present invention, said region of the patient is a vitreous of an eye.

In yet another aspect, the pH of the buffer remains within about 0.1 pH unit of the original buffer pH.

In one aspect, the buffer has a pH of about 7. Alternatively, the buffer has a pH in a range from about 7 to about 7.5.

In another aspect, the buffer has a pH of about 7.4.

In still another aspect, the buffer is a phosphate buffer or a Tris-HCl buffer. In yet another aspect, the buffer is 10× phosphate buffered saline (“PBS”) or 5× PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propanesulfonic acid) having pKₐ of 10.4 at 25° C. and pH in the range of about 9.7-11.1.

### EXAMPLE 1

### Plasmin Precipitation Study

Sterile, purified, and unbuffered human plasmin (pH of 3.3±0.3) in a stable, lyophilized form and without any preservative was obtained from Talecris, Inc. (Research Triangle Park, North Carolina).

Plasmin (100 μg (equivalent to ˜4.7 IU/50 μl) was mixed in a 1:4 ratio with 10× PBS solution (pH of 7.4, obtained from Invitrogen, Carlsbad, Calif.). Normal saline was used instead of PBS for control. An amount of 50 μl of each mixture was then injected separately into the central area of 1 ml of clear homogenized porcine vitreous at 37° C., and observation for any precipitation at room temperature was made. The control (saline-based plasmin) formulation showed rapid precipitation within 3 minutes while the 10× or the 5× formulation did not precipitate. Thus, acidified plasmin reconstituted in a strong buffer having near-neutral pH substantially immediately prior to injection into a near-neutral medium avoided precipitation.

### EXAMPLE 2

### Buffered Plasmin Activity

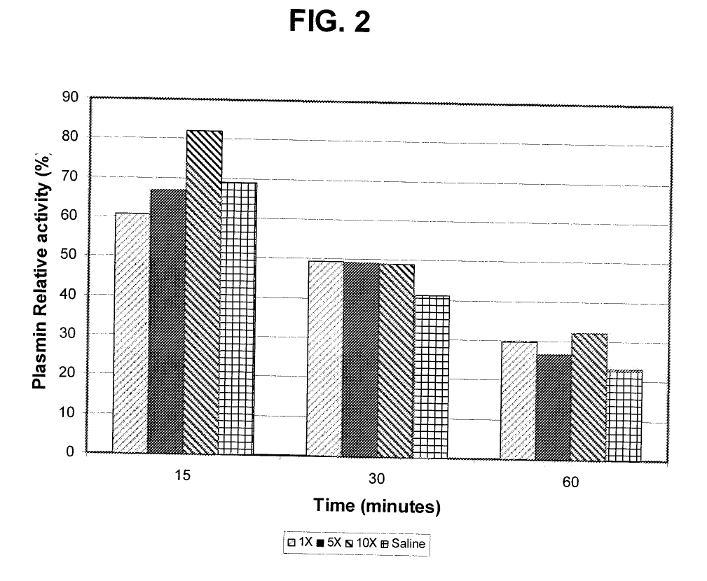

Plasmin (100 μg (equivalent to ˜4.7 IU)/50 μl) was mixed in a 1:4 ratio with 10×, 5×, and 1× PBS solution. An amount of 50 μl of each combination was added to 1 ml clear homogenized porcine vitreous, mixed thoroughly, and the mixture was incubated at 37° C. Plasmin activity was measured using the S-2251 chromogenic assay at time t=0, 15, 30, and 60 minutes. S-2251 is a short peptide substrate for plasmin (H-D-Val-L-Leu-L-Lys-p-nitroaniline dihydrochloride, available from Chromogenix-lnstrumentation Laboratory SpA, Milano, Italy). Plasmin hydrolyzes this substrate between the lysine residue and the p-nitroaniline moiety. The method determines the activity of plasmin based on the difference in absorbance (optical density) between the p-nitroaniline formed and the original substrate. The rate of p-nitroaniline formation; i.e., the increase in absorbance per second at wavelength of 405 nm, is proportional to the enzymatic activity of plasmin, and is conveniently measured with a photometer. The activity of plasmin relative to initial activity is shown in FIG. 2. Plasmin reconstituted in 10× PBS solution favorably retained its activity compared to the control sample (plasmin reconstituted in only saline solution). In addition to precipitation in the control sample, as observed similarly in Example 1, there was some precipitation for the sample reconstituted with 1× PBS solution. It should be noted that alternate chromogenic substrates for plasmin also may be used to determine its enzymatic activity, such as S-2390 (H-D-Val-L-Phe-L-Lys- p-nitroaniline dihydrochloride) or S-2403 (L-Pyroglutamyl-L-Phe-L-Lys-p-nitroaniline dihydrochloride); both are available from Chromogenix-lnstrumentation Laboratory SpA, Milano, Italy.

Another aqueous buffer comprising 0.185 weight percent of anhydrous sodium phosphate monobasic, 0.98 weight percent of anhydrous sodium phosphate dibasic, and 0.4 weight percent of sodium chloride, having a pH of about 7.4 and osmolarity of 340 mOsm/l also was tested. The reconstituted plasmin pH was about 7.2. The results obtained with this buffer solution were similar to those obtained with 10× PBS solution. It should be noted that osmolarity in the range from about 200-350 mOsm/l can be equally applicable for use in a method of the present invention.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for producing an active enzyme after prolonged storage, the method comprising:
(a) preparing said enzyme;
(b) storing said enzyme at a pH less than about 5; and
(c) adding said enzyme to a buffer having pH corresponding approximately to a pH at which said enzyme has highest activity, to produce a buffered enzyme substantially immediately before use;
wherein said enzyme is autodegradable at pH greater than about 5; and said buffer has a capacity such that a pH of a buffered enzyme solution remains within about 1 pH unit upon adding said enzyme.

2. The method of claim 1, the pH of the buffered enzyme solution remains within about 0.5 pH unit upon adding said enzyme.

3. The method of claim 1, the pH of the buffered enzyme solution remains within about 0.2 pH unit upon adding said enzyme.

4. The method of claim 1, wherein said enzyme is a proteolytic enzyme.

5. The method of claim 1, wherein said enzyme is selected from the group consisting of serine proteinases, cysteine proteinases, aspartyl proteinases, metalloproteinases, and combinations thereof.

6. The method of claim 1, wherein said enzyme is plasmin or a plasmin derivative.

7. A method for prolonging an activity of an enzyme at physiological pH, which enzyme is autodegradable at said physiological pH, the method comprising:
(a) providing said enzyme that have been preserved at a pH less than about 5; and
(b) adding said enzyme to a buffer having approximately physiological pH to produce a buffered enzyme before administering said buffered enzyme into a patient, thereby prolonging the activity of said enzyme in said patient; wherein the post-administering activity in said patient is higher than the activity of unbuffered enzyme, and said buffer has a capacity such that a pH of buffered enzyme solution remains within about 1 pH unit upon adding said enzyme.

8. A method for prolonging an activity of plasmin or derivatives thereof in a posterior chamber of an eye, the method comprising:
(a) providing said plasmin or derivatives thereof that have been preserved at a pH less than about 5; and
(b) adding said plasmin or derivatives thereof to a buffer having a pH in a range from about 6.5 to about 11 to produce a buffered plasmin or derivatives thereof before administering said buffered plasmin or derivatives thereof into said posterior chamber of the eye, thereby prolonging the activity of plasmin or derivatives thereof in said posterior chamber of the eye; wherein the post-administering activity is higher than the activity of unbuffered plasmin or derivatives thereof in said posterior chamber of the eye, and said buffer has a capacity such that a pH of buffered solution of said plasmin or derivatives thereof remains within about 1 pH unit upon adding said plasmin or derivatives thereof.

9. The method of claim 8, wherein precipitation of said plasmin or derivatives thereof in said posterior chamber of the eye is avoided upon administering said plasmin or derivatives thereof.

10. The method of claim 8, a pH of a buffered enzyme solution remains within about 0.1 pH unit upon adding said enzyme.

11. A method for inducing posterior vitreous detachment (“PVD”) in an eye, the method comprising:
(a) providing plasmin or derivatives thereof that have been preserved at a pH less than about 5; and
(b) adding said plasmin or derivatives thereof to a buffer having a pH in a range from about 6.5 to about 11 to produce a buffered plasmin or derivatives thereof before administering said buffered plasmin or derivatives thereof into a posterior chamber of the eye, thereby inducing PVD in said eye, wherein said buffer has a capacity such that a pH of a buffered solution of said plasmin or derivatives thereof remains within about 1 pH unit upon adding said plasmin or derivatives thereof.

12. The method of claim 11, wherein precipitation of said plasmin or derivatives thereof in said posterior chamber of the eye is avoided upon administering said plasmin or derivatives thereof.

13. The method of claim 11, wherein said plasmin or derivatives thereof have been preserved at pH in a range from about 2.5 to about 4.

14. The method of claim 11, a pH of a buffered enzyme solution remains within about 0.5 pH unit upon adding said enzyme.

15. A method for preventing or reducing a precipitation of an enzyme administered into a region of a patient, the method comprising:
(a) providing the enzyme at a pH of less than about 5;
(b) adding said enzyme to a buffer having a pH in a range from about 6.5 to about 11 to produce a buffered enzyme before administering said buffered enzyme into said region of the patient; wherein upon adding the enzyme to the buffer, the pH of the buffer remains within about 1 pH unit of the pH of the buffer.

16. The method of claim 15, wherein said region of the patient is a vitreous body of an eye.

17. The method of claim 15, wherein the pH of the enzyme of step (a) is in a range from about 2.5 to about 4.

18. The method of claim 15, a pH of a buffered enzyme solution remains within about 0.1 pH unit upon adding said enzyme.

19. The method of claim 15, wherein said enzyme is a proteolytic enzyme.

20. The method of claim 15, wherein said enzyme is selected from the group consisting of serine proteinases, cysteine proteinases, aspartyl proteinases, metalloproteinases, and combinations thereof.

21. The method of claim 15, wherein said enzyme is plasmin or plasmin derivatives.

22. The method of claim 14, wherein said region of a patient is a circulatory system of said patient.

23. A kit for making an active enzyme or derivatives thereof, said kit comprising: (a) the enzyme or derivatives thereof that have been preserved at a pH less than about 5; and (b) a buffer having a pH in a range from about 6.5 to about 11, provided in a separate container or package.

24. The kit of claim 23, wherein said enzyme is plasmin or derivatives thereof.

25. The kit of claim 24, wherein said plasmin or derivatives thereof have been preserved at a pH in a range from about 3 to 4.

26. The kit of claim 24, wherein said buffer has a capacity such that a pH of a buffered solution of said plasmin or derivatives thereof remains within about 1 pH unit upon adding said plasmin or derivatives thereof.

27. A method of for inducing PVD in an eye, the method comprising administering a formulation of plasmin or derivatives thereof into a posterior chamber of an eye of a patient in need of having PVD; wherein said plasmin or derivatives thereof have been preserved at a pH less than about 5; and the preserved plasmin or derivatives thereof have been added to a buffer having a pH in a range from about 6.5 to about 11 to produce said formulation of plasmin or derivatives thereof before administering said formulation into a posterior chamber of the eye, thereby inducing PVD in said eye, wherein said buffer has a capacity such that a pH of a buffered solution of said plasmin or derivatives thereof remains within about 1 pH unit upon adding said plasmin or derivatives thereof, and said formulation is administered in an amount containing a therapeutically effective amount of plasmin or derivatives thereof to induce said PVD.

28. The method of claim 27, wherein said plasmin or derivatives thereof are added to said buffer substantially immediately before said administering.
